(19) United States
(12) Patent Application Publication
(10) Pub. No.: US 2013/0109929 A1
(43) Pub. Date: May 2, 2013

(54) SYSTEMS AND METHODS FOR PATIENT MONITORS TO AUTOMATICALLY IDENTIFY PATIENTS

(71) Applicant: Frank Menzel, Oakland, NJ (US)

(72) Inventor: Frank Menzel, Oakland, NJ (US)

(73) Assignee: MINDRAY DS USA, INC., Mahwah, NJ (US)

(21) Appl. No.: 13/284,333

(22) Filed: Oct. 28, 2011

(51) Int. Cl. A61B 5/00

(52) U.S. Cl. 600/301

(57) ABSTRACT

A portable patient monitoring system may be configured to automatically identify a patient using remote sensing technology, such as radio frequency identification (RFID) or Long Wavelength ID (LWID). A patient monitoring system, such as a portable spot-checking monitor, may be brought in proximity with a patient in order for a remote sensor to receive identification information from an electronic identification device (EID), such as an RFID tag or an LWID tag, associated with the patient. The patient monitoring system may acquire data signals relating to the patient's physiological parameters. The patient monitoring system may process the data signals and generate patient parameter information. The patient monitoring system may then associate the patient parameter information with the identification information and store the information within a memory unit, display the information, and/or upload the information. The patient monitoring system may retrieve a patient identity using the identification information received from the EID.

### TECHNICAL FIELD

This disclosure relates to patient monitors. Specifically, this disclosure relates to portable patient monitoring systems configured to automatically identify patients.

### SUMMARY

According to various embodiments, a patient monitoring system, such as a portable spot-checking monitor, may automatically identify a patient using radio frequency identification (RFID). In one embodiment, a patient monitoring system may be brought in proximity with a patient. An RFID reader in communication with the patient monitoring system may receive identification information from an RFID tag associated with the patient. The patient monitoring system may acquire data signals relating to various physiological parameters of the patient. For example, the patient monitoring system may be used to determine or record a patient's blood pressure, heart rate, temperature, and/or other physiological parameters. The patient monitoring system may process the data signals and generate patient parameter information. The patient monitoring system may then associate the patient parameter information with the identification information and store the information within a memory unit, display the information on a display unit, and/or upload the information to a central management system. According to some embodiments, a patient monitoring system may retrieve a patient identity using the identification information received from the RFID tag associated with the patient. The patient monitoring system may request that an operator verify that the retrieved patient identity corresponds to the patient actually being monitored. Additional aspects will be apparent from the following detailed description, which proceeds with reference to the accompanying drawings.

### BRIEF DESCRIPTION OF THE DRAWINGS

FIG. 1 illustrates a block diagram of a patient monitoring system.

FIG. 2 is a perspective view of a patient monitoring system, including an integrated radio frequency identification (RFID) reader, a display, and various communication ports.

FIG. 3A is a perspective view of a patient monitoring system, including a barcode scanner, a display, and various communication ports.

FIG. 3B is a perspective view of a patient monitoring system, including a handheld RFID reader, a display, and various communication ports.

FIG. 4 is a perspective view of a portable patient monitoring system secured to a stand and rolling base.

FIG. 5A is a perspective view of a portable patient monitoring system with an integrated RFID reader in communication with an RFID tag secured to a bed of a patient.

FIG. 5B is a perspective view of a portable patient monitoring system with a handheld RFID reader in communication with an RFID tag embedded in a bracelet of a patient.

FIG. 5C is a perspective view of a portable patient monitoring system with a handheld RFID reader in communication with an RFID tag secured to a patient identification disk.

FIG. 6 illustrates a handheld RFID reader being used to scan an RFID tag embedded in a bracelet secured to the wrist of a patient, according to one embodiment.

FIG. 7 is a functional block diagram of a computer system that may be used to monitor a patient and automatically detect the identity of a patient using RFID.

FIG. 8 is a flow chart of an exemplary method for automatically associating patient parameter information with identification information received via an RFID tag associated with a patient.

FIG. 9A is a flow chart of an exemplary method for automatically identifying and verifying a patient identity using an RFID tag.

FIG. 9B is a flow chart of an exemplary method continued from FIG. 9A for automatically associating patient parameter information with the patient identity using an RFID tag.

### DETAILED DESCRIPTION

Patient monitoring systems may be used to analyze and display physiological parameters obtained from sensors attached to a patient. The physiological parameters may include, for example, pulse, temperature, respiration, blood pressure, blood oxygen, electrocardiogram, etc. Patient monitoring systems may be configured to function as portable spot-checking devices moved between various rooms in a medical facility to perform routine physiological monitoring of multiple patients. A patient monitoring system, as described herein, may be configured to automatically determine the identity of a patient prior to or while measuring the physiological parameters of the patient.

According to various embodiments, a patient monitoring system configured to automatically determine identification information associated with a patient and/or a patient identity may eliminate the need for an operator to manually input such information prior to acquiring data signals related to the patient parameters. Moreover, the patient parameter information may be automatically associated with a patient and electronically uploaded to a central management system, eliminating the need to print the data from a patient monitoring device and/or to manually enter it into the central management system.

In one embodiment, a patient monitoring system includes a display unit, a parameter acquisition unit, and a remote sensor. While the present disclosure primarily refers to RFID technology, a skilled artisan will recognize that other remote sensing technologies may be used, such as LWID (Long Wavelength ID), also known as RuBee or IEEE 1902.1, VLID (Visible Light ID), Skinplex (available from Ident Technology AG), Microfiber Sensor Tracking (available from Demodulation, Inc.), near field communication (NFC), Bluetooth wireless technologies, etc.

The patient monitoring system may use the remote sensor to receive identification information from an electronic identification device (EID) associated with the patient. For example, an EID may comprise an RFID tag, a LWID tag, an identification device in a Skinplex or Microfiber Sensor Tracking system. In some embodiments an EID may comprise programmable electronic components allowing the EID to be reprogrammed. For example, an EID may be reprogrammed to communicate new identification information each time the EID is associated with a new patient. Accordingly, in some embodiments, an EID may include a memory, a microcontroller, and/or other programmable electronic components. Alternatively, the EID may be pre-programmed during manufacturing, such as may be the case with an RFID tag.

The remainder of this disclosure will refer more particularly to embodiments utilizing RFID, recognizing that other technologies may be used within the scope of the disclosure. In such embodiments, the remote sensor is an RFID reader and the EID is an RFID tag. The RFID reader may be integrated into the patient monitoring system or a peripheral device attached to, or in communication with, the patient monitoring system.

The parameter acquisition unit may acquire data signals relating to physiological parameters of the patient. After processing the signals, the patient monitoring system may generate patient parameter information relating to the physiological parameters based on the data signals. The patient monitoring system may then associate the patient parameter information with the identification information and store it in memory. The patient monitoring system may display some or all of the data signals, the identification information, and/or the patient parameter information on a display unit.

According to various embodiments, the identification information received may comprise a unique number transmitted by the RFID tag to the RFID reader. A database may be maintained within a central database and/or within the patient monitoring system associating the unique number with a medical facility room, a medical facility bed, patient clothing, a location within the medical facility, an object/fixture within the medical facility, and/or a patient bracelet. Further, the database and/or the patient monitoring system may associate the medical facility bed, room, object, and/or bracelet with a current or past patient.

The identification information obtained from an RFID tag associated with a patient may be used to automatically retrieve a patient identity. The database of patient identities may be locally stored on the patient monitoring system and/or remotely stored. The patient monitoring system may then request that an operator, such as a doctor, nurse, or technician, verify that the patient identity corresponds to the patient about to be monitored.

In some embodiments, the retrieved patient identity may not be fully provided to the operator. Rather, only a portion of the patient identity may be provided to the operator for verification purposes. For example, the patient monitoring system may utilize the identification information to obtain a patient identity that includes only a first name, a last name, a portion of a birth date, and/or a personal identification number (PIN). This may allow for the verification of the retrieved patient identity while minimizing the exposure of a patient's personal information. Once at least a portion of the patient identity has been automatically retrieved, the patient monitoring system may request that an operator verify that the provided portion of the patient identity corresponds to the patient actually being monitored.

If the verification fails, then the RFID reader may attempt to determine a patient identity again. Alternatively or in addition, an operator may manually input the patient's identity. Once the patient has been correctly identified, the patient monitoring system may acquire data signals relating to physiological parameters of the patient, process the signals, and generate patient parameter information. The patient monitoring system may then associate the patient parameter information with the identification information and/or the patient identity and store the information in memory. The patient monitoring system may display some or all of the data signals, the identification information, the patient identity, and/or the patient parameter information on a display unit, such as a touch screen.

The patient monitoring system may utilize a wireless network connection to retrieve the patient identity from a remote database. Additionally, the patient monitoring system may utilize a wireless network connection to upload the identification information, the patient identity, and/or the associated patient parameter information to a central management system, such as a server or centralized database.

A barcode scanner may be utilized to determine a patient's identity in addition to, or in place of, an RFID reader. A barcode scanner may be used to scan a barcode associated with a room of a medical facility, a bed within a medical facility, an object within the medical facility, and/or a bracelet on a patient. The scanned barcode may be used to identify a patient and/or to provide verification that an RFID reader has correctly identified the patient using an RFID tag. For example, an RFID reader may receive identification information from an RFID tag secured to the bed of a patient. The patient monitoring system may retrieve a patient identity corresponding to the received identification information. A barcode scanner may then be used to scan a bracelet attached to the wrist of a patient. The resulting barcode scan may be used to verify that the patient has been properly identified.

Some of the infrastructure that can be used with embodiments disclosed herein is already available, such as: general-purpose computers, RFID tags, RFID readers, computer programming tools and techniques, digital storage media, and communications networks. A computer may include a processor such as a microprocessor, microcontroller, logic circuitry, or the like. The processor may include a special purpose processing device such as an ASIC, PAL, PLA, PLD, FPGA, or other customized or programmable device. The computer may also include a computer-readable storage device such as non-volatile memory, static RAM, dynamic RAM, ROM, CD-ROM, disk, tape, magnetic, optical, flash memory, or other computer-readable storage medium.

Various aspects of certain embodiments may be implemented using hardware, software, firmware, and/or a combination thereof. As used herein, a software module or component may include any type of computer instruction or computer executable code located within or on a computer-readable storage medium. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media. Some embodiments may be practiced in a distributed computing environment where tasks are performed by a remote processing device linked through a communications network.

As used throughout this disclosure, an RFID tag is a generic term relating to a device that wirelessly transmits a unique identifying number. An RFID tag may comprise an active RFID tag, a battery assisted passive (BAP) RFID tag, or a passive RFID tag. An active RFID tag may be independently powered, such as by light or a battery. An active RFID tag may continually transmit a signal or may transmit a signal once a receiver has been successfully identified. A BAP RFID tag may require an external stimulus, such as light or an electromagnetic field, in order to wake up prior to transmitting a signal. A passive RFID tag may not require a power source and may require an external stimulus, such as an electromagnetic field, to initiate a signal transmission.

In the exemplary embodiments depicted in the drawings, the size, shape, orientation, placement, configuration, and/or other characteristics of RFID tags and RFID readers are merely illustrative. Specifically, RFID tags are commonly manufactured very small and may not necessarily be as obtrusive as depicted in the drawings. Additionally, RFID readers may be of any shape and/or size and be portable, handheld, and/or stationary, and are not limited by the exemplary illustrations. Moreover, the RFID readers, which may be significantly smaller than illustrated, may be less intrusively placed and/or configured differently from those depicted in the drawings.

The embodiments of the disclosure will be best understood by reference to the drawings, wherein like parts are designated by like numerals throughout. The components of the disclosed embodiments, as generally described and illustrated in the figures herein, could be arranged and designed in a wide variety of different configurations. Furthermore, the described features, structures, or operations may be combined in any suitable manner in one or more embodiments. In other instances, well-known structures, materials, or operations are not shown or described in detail to avoid obscuring aspects of this disclosure.

Thus, the following detailed description of the embodiments of the systems and methods of the disclosure is not intended to limit the scope of the disclosure, as claimed, but is merely representative of possible embodiments. In addition, the steps of a method do not necessarily need to be executed in any specific order, or even sequentially, nor need the steps be executed only once.

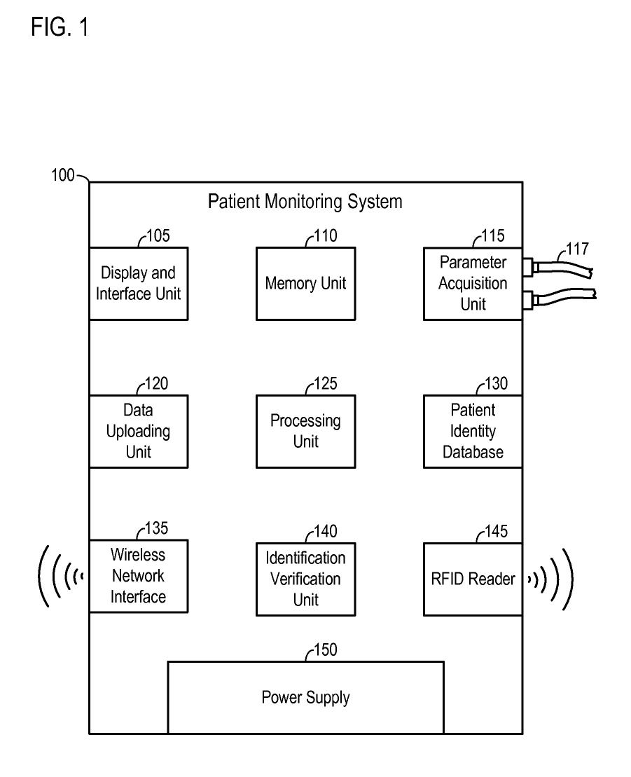

FIG. 1 illustrates a block diagram of a patient monitoring system 100, according to various embodiments. As illustrated, patient monitoring system 100 may include a display and interface unit 105, a memory unit 110, a parameter acquisition unit 115, a data uploading unit 120, a processing unit 125, a patient identity database 130, a wireless network interface 135, an identification verification unit 140, an RFID reader 145, and/or a power supply 150. According to various embodiments, a patient monitoring system may include more or less functionality than the illustrated embodiment. According to some embodiments, a patient monitoring system may be configured as a portable patient monitor and utilize a rechargeable power supply 150.

RFID reader 145 may be configured to receive identification information from an RFID tag. RFID reader 145 may be configured to receive identification information from active, BAP, and/or passive RFID tags. Accordingly, RFID reader 145 may be configured as an RFID interrogator transmitting a signal and receiving a response signal or as a passive listening device. RFID reader 145 may be configured as a fully integrated device within patient monitoring system 100, integrated as a physical protrusion to patient monitoring system 100, and/or as a peripheral device in communication with patient monitoring system 100 via a wire or wireless connection.

Parameter acquisition unit 115 may utilize connectors 117 to acquire one or more data signals relating to various physiological parameters of a patient. For example, parameter acquisition unit 115 may acquire data signals relating to pulse, temperature, respiration rate, blood pressure, venous oxygen saturation, electrocardiogram information, and the like. Processing unit 125 may be configured to process the data signals in order to generate patient parameter information relating to the physiological parameters based on the acquired data signals. Processing unit 125 may then associate the patient parameter information with the identification information and store it within memory unit 110.

Accordingly, memory unit 110 may contain identification information for one or more patients and associated patient parameter information for each of the patients. An operator may access memory unit 110 in order to obtain the patient parameter information for a specific patient. Additionally, data uploading unit 120 may upload data within memory unit 110, including the identification information of various patients and the associated patient parameter information, to a centralized database or central management system. For example, data uploading unit 120 may upload data from within memory unit 110 to a server using a wired connection after patient monitoring system 100 has been used to monitor one or more patients. Alternatively, data uploading unit 120 may wirelessly upload the identification information of various patients and the associated patient parameter information at regular intervals, on demand, or whenever a wireless connection is made available.

Patient monitoring system 100 may include a local patient identity database 130, or alternatively be configured to wirelessly query a remote patient identity database. Patient monitoring system 100 may retrieve a patient identity from patient identity database 130 using the identification information received by RFID reader 145. According to various embodiments, the patient identity may include the first and last name of a patient and/or other personal identification data related to a patient. For example, a patient identity may include a first name, a last name, a portion of a birth date, and/or a PIN.

Patient monitoring system 100 may also include an identification verification unit 140 configured to prompt an operator to verify that a patient identity retrieved from patient identity database 130 actually matches the patient being monitored. According to some embodiments, the retrieved patient identity may not be fully provided to the operator. Rather, only a portion of the patient identity may be provided to the operator for verification purposes. For example, the patient monitoring system 100 may utilize the identification information to obtain a patient identity that includes only a first name, a last name, a portion of a birth date, and/or a PIN. This may allow for verification while minimizing the exposure of a patient's personal information.

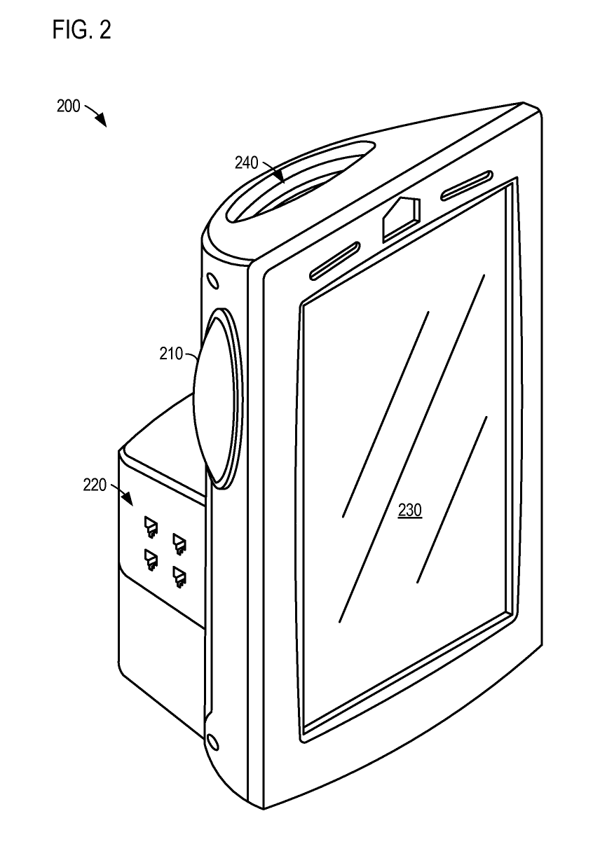

FIG. 2 illustrates an exemplary embodiment of a patient monitoring system 200, including an integrated RFID reader 210, a display 230, and various communication ports 220. A patient monitoring system may be configured to have any shape, size, and/or dimensions. As illustrated, a handle 240 may provide a location to grip patient monitoring system 200. RFID reader 210 is illustrated as a protrusion on a side of patient monitoring system 200. According to alternative embodiments, an RFID reader 210, or a portion thereof, may be formed as a protrusion anywhere on patient monitoring system 200. Alternatively, RFID reader 210 may be completely integrated within the casing of patient monitoring system 200.

Communication ports 220 may include any of a wide variety of port types and sizes. For example, communication ports 220 may include network ports, such as RJ-45 Ethernet ports and RS-232 ports, nurse call ports, coaxial ports, and/or specialized ports for connecting physiological sensor probes, such as SpO₂ ports. Display 230 may include an integrated touch screen allowing for operator input. Alternatively or additionally, patient monitoring system 200 may include one or more peripheral devices, such as a keyboard or mouse, configured to allow for operator inputs.

As illustrated in FIG. 2, display 230 may have a vertical dimension larger than a horizontal dimension. In certain embodiments, orienting display 230 in a portrait orientation allows for a larger number of vertically aligned rectangular regions, and thus a larger number of patient parameter waveforms, to be displayed than if display 230 were oriented in a landscape orientation. Particularly, as orienting display 230 in a portrait orientation allows for an increased vertical dimension, more vertically aligned waveforms may be displayed on the interface. While orienting display 230 in a portrait orientation may reduce the length of the horizontally displayed time parameters of the patient parameter waveforms, the added benefit of displaying a greater number of patient parameter waveforms may be beneficial to a clinical practitioner.

In certain embodiments, display 230 included in patient monitoring system 200 may have omni-directional visibility and be capable of being viewed from a wide variety of angles. In some embodiments, backlight inverters (not shown) included in display 230 may be oriented in a manner allowing for their normal operation (e.g., horizontally). In other embodiments, display 230 may be lit using light-emitting diodes (LEDs).

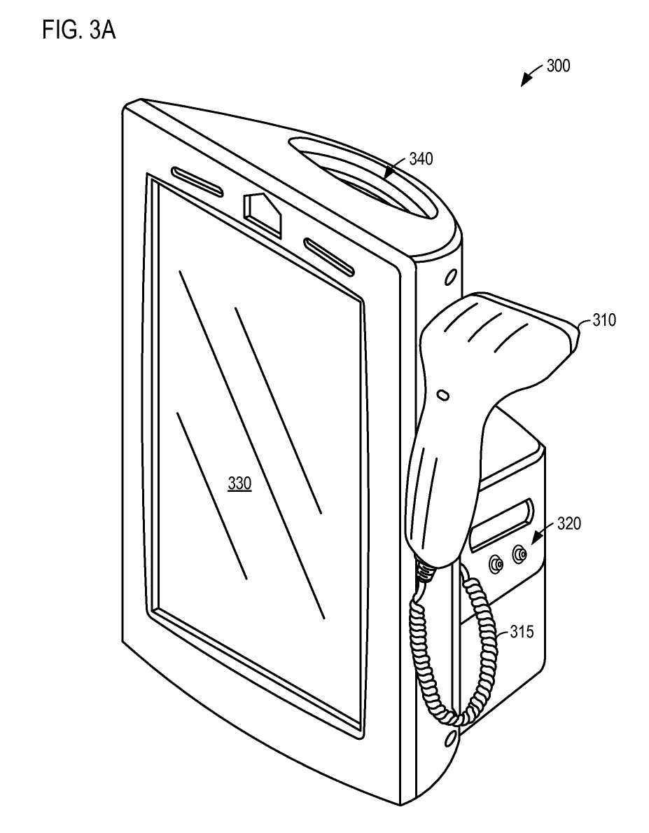

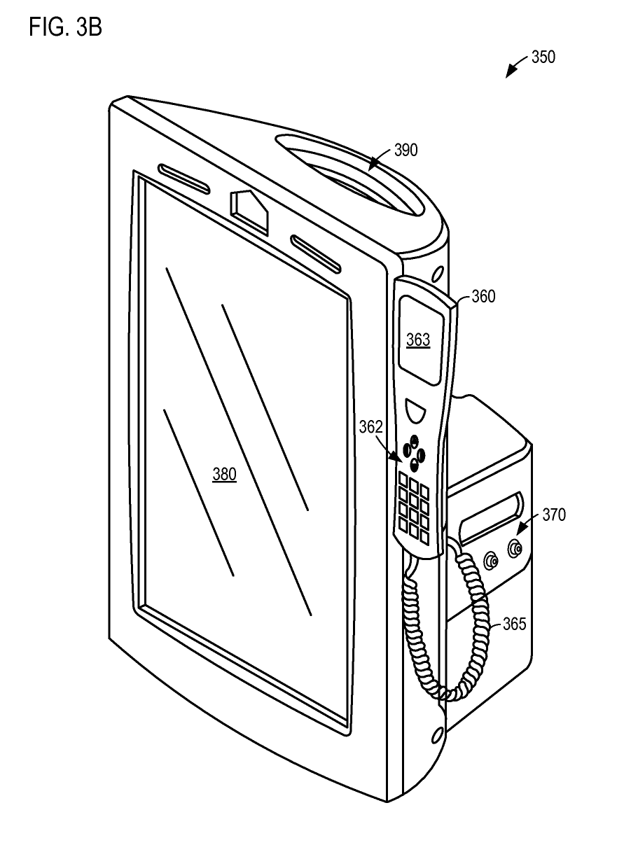

FIG. 3A illustrates an exemplary embodiment of a patient monitoring system 300, including a barcode scanner 310, a display 330, and various communication ports 320. As illustrated, a handle 340 may provide a grip for the movement or transportation of patient monitoring system 300. Barcode scanner 310 may be in communication with patient monitoring system 300 via a coiled cable 315. Alternatively, barcode scanner 310 may communicate wirelessly with patient monitoring system 300. Patient monitoring system 300 may include various communication ports 320, such as network ports, nurse call ports, and sensor ports. For example, patient monitoring system 300 may include coaxial ports, RS-232 ports, RJ-45 ports, and/or SpO₂ ports.

Display 330 may include a touch screen configured to receive inputs from an operator. Additionally, one or more peripheral devices in addition to barcode scanner 310 may be included to allow for additional operator input. The exact size and orientation of display 330 may be adapted to suit a particular need.

FIG. 3B illustrates an exemplary embodiment of a patient monitoring system 350, including a handheld RFID reader 360, a display 380, and various communication ports 370. Again, communication ports 370 may include any of a wide variety of port types and sizes appropriate for networking, remote monitoring, and/or connecting physiological sensors. A handle 390 may facilitate the transportation of patient monitoring system 350. Display 380 may comprise a touch panel or touch sensitive screen.

As illustrated, a handheld RFID reader 360 may be in communication with patient monitoring system 350 via a coiled cable 365. Alternatively, handheld RFID reader 360 may communicate wirelessly with patient monitoring system 350. Handheld RFID reader 360 may include various buttons 362 and/or a display screen 363. Alternatively, RFID reader 360 may comprise a wand with no buttons or display. Handheld RFID reader 360 may be formed in any suitable shape or size. According to some embodiments, patient monitoring system 350 may include an integrated RFID reader, as illustrated in FIG. 2, and/or a barcode scanner, as illustrated in FIG. 3A, in addition to handheld RFID reader 360.

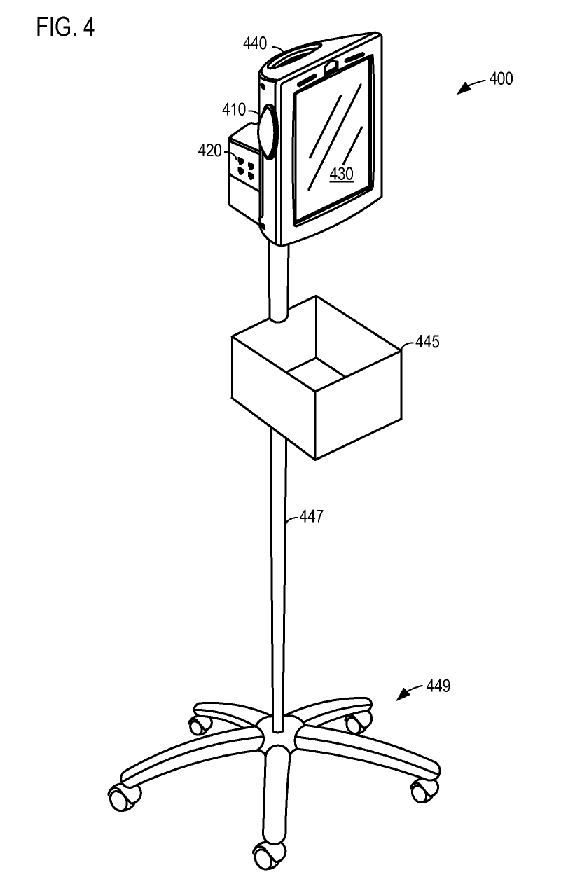

FIG. 4 illustrates an exemplary embodiment of a portable patient monitoring system 400 secured to a stand 447 and a rolling base 449. According to various embodiments, portable patient monitoring system 400 may include a handle 440, a display 430, various ports 420, and/or an integrated RFID reader 410. As described in conjunction with other embodiments, ports 420 may include network ports, nurse call ports, communication ports, monitoring ports, and/or physiological sensor ports. Display 430 may comprise a touch screen display. Portable patient monitor 400 may include a barcode scanner, a handheld RFID reader, and/or other peripheral devices in addition to or in place of integrated RFID reader 410. Stand 447 and rolling base 449 may be replaced with any of a wide variety of suitable stands and/or bases. Stand 447 may include one or more storage containers, such as storage container 445.

Portable patient monitoring system 400 may be configured as a portable handheld spot-checking device. In such an embodiment, the size of display 430 and of the surrounding case may be greatly reduced and not require stand 447 or rolling base 449. Additionally, wireless connections suitable for each of the various peripheral devices and physiological sensors may replace ports 420. The embodiment shown in FIG. 4 is provided by way of example, and a skilled artisan will understand from the disclosure that any portable patient monitoring system may be used with the embodiments disclosed herein.

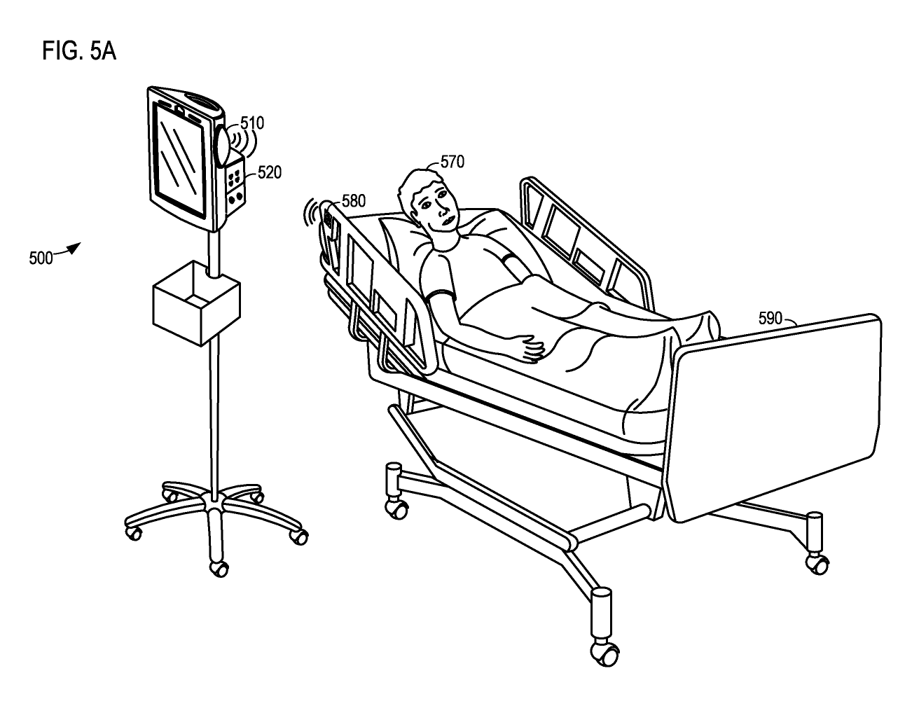

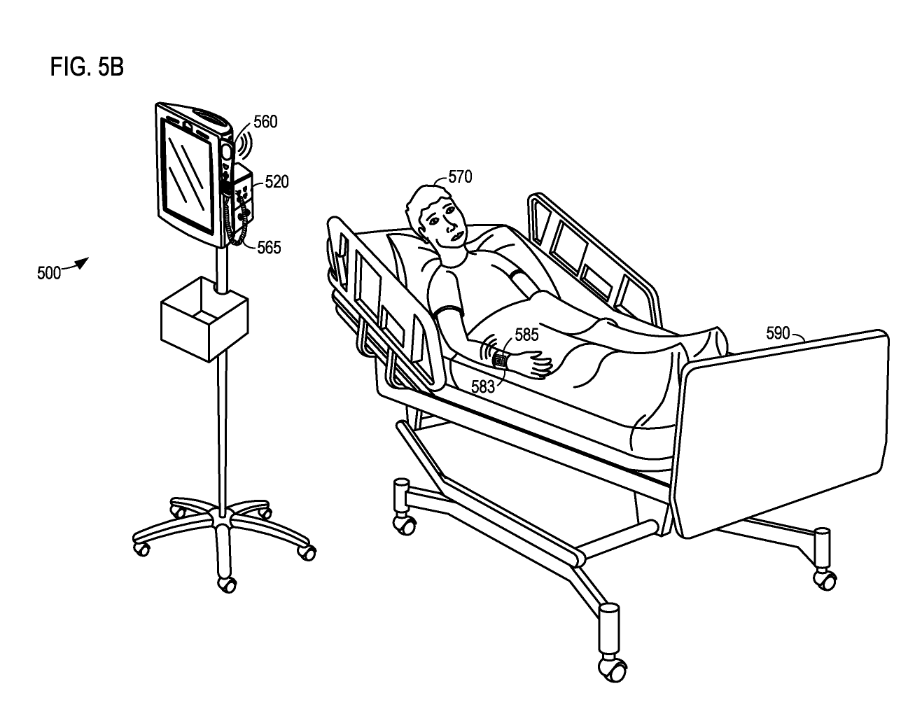

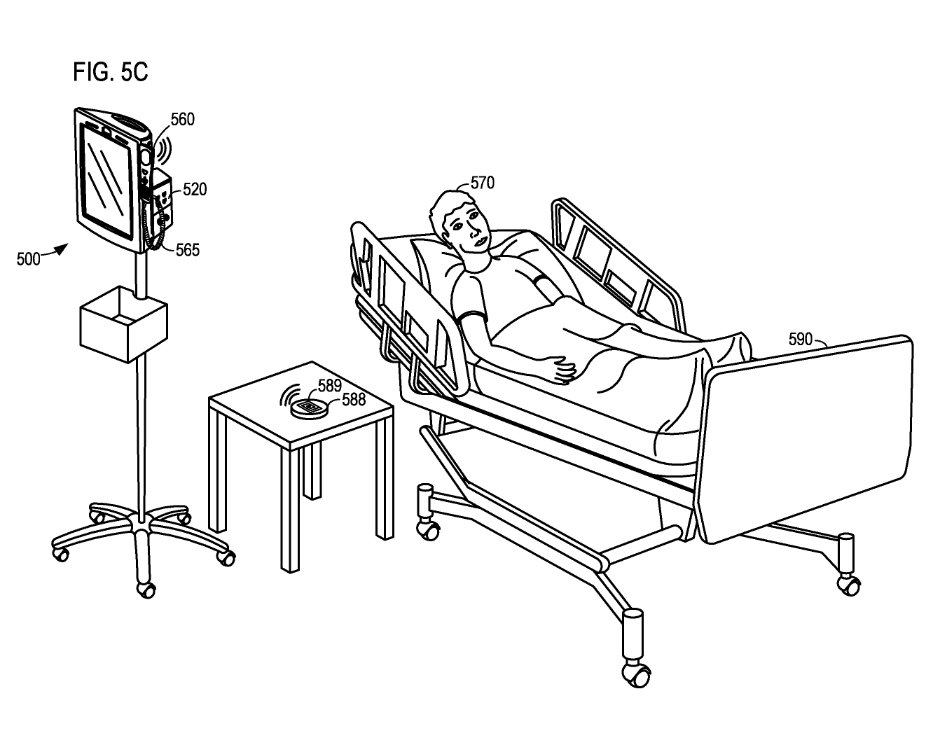

FIG. 5A illustrates a portable patient monitoring system 500 with an integrated RFID reader 510 in communication with an RFID tag 580 secured to the bed 590 of a patient 570, according to one embodiment. As illustrated, a portable patient monitoring system 500 may be brought near patient 570 in order to monitor various physiological parameters of patient 570. Integrated RFID reader 510 may be configured to communicate with an RFID tag 580 associated with patient 570. Integrated RFID reader 510 may be a passive listening RFID reader or an active RFID interrogator. Similarly, RFID tag 580 may comprise a passive, BAP, and/or an active battery-powered RFID tag.

Integrated RFID reader 510 may receive identification information from RFID tag 580 associated with patient 570. According to some embodiments, portable patient monitoring system 500 may retrieve the patient identity of patient 570 using the received identification information. The patient identity corresponding to the identification information received from RFID tag 580 may be stored in a local database within portable patient monitoring system 500 or made accessible to portable patient monitoring system 500 via a wireless network connection. Portable patient monitoring system 500 may then request that an operator verify that the retrieved patient identity corresponds to patient 570. Ports 520 may be utilized to connect various physiological sensors (not shown) to portable patient monitoring system 500. The various physiological sensors may be used to acquire data signals related to physiological parameters of patient 570.

For example, ports 520 may be used to acquire data signals relating to pulse, temperature, respiration rate, blood pressure, venous oxygen saturation, electrocardiogram information, and the like. Portable patient monitoring system 500 may be configured to process the data signals in order to generate patient parameter information relating to the physiological parameters of patient 570. Portable patient monitoring system 500 may associate the patient parameter information with the identification information and/or the patient identity and store it within memory. Alternatively or additionally, portable patient monitoring system 500 may upload the identification information, the associated patient parameter information, and/or the patient identity via a wireless network connection to a central management system.

As illustrated in FIG. 5A, RFID tag 580 associated with patient 570 may be secured to bed 590 of patient 570. The identification information transmitted by RFID tag 580 to integrated RFID reader 510 may be temporarily associated with the patient identity of patient 570 for as long as patient 570 is assigned bed 590. Once a new patient is moved into bed 590, the identification information provided by RFID tag 580 may be re-associated with the new patient.

As illustrated in FIG. 5B, a portable patient monitoring system 500 may include a handheld RFID reader 560 configured to receive identification information from an RFID tag 585 secured to or embedded in a bracelet 583 attached to a wrist or ankle of patient 570. Handheld RFID reader 560 may remain selectively secured to a side of portable patient monitoring system 500 and still be able to receive identification information from RFID tag 585. Alternatively, handheld RFID reader 560 may be selectively removed from the side of portable patient monitoring system 500 in order to receive identification information from RFID tag 585. Handheld RFID reader 560 may remain in communication with portable patient monitoring system 500 via coiled cable 565 or via a wireless connection. Handheld RFID reader 560 may be brought relatively close to RFID tag 585 by an operator, reducing the required signal strength of RFID tag 585. According to some embodiments, the use of handheld RFID readers may allow for the use of limited range RFID tags in order to prevent cross-reads with other nearby RFID tags.

FIG. 5C illustrates a portable patient monitoring system 500 with a handheld RFID reader 560 attached via a coiled cable 565. Handheld RFID reader 560 may be configured to receive identification information from an RFID tag 589 secured or embedded in a patient identification disk 588 associated with patient 570. RFID tag 589 may be secured to or embedded in any of a wide variety of objects configured to be carried by patient 570. According to some embodiments, RFID tag 589 may be permanently associated with patient 570. Patient 570 may then take an object, such as patient identity disk 588, from a medical facility and bring it back during subsequent visits. According to one embodiment, an RFID tag may be embedded in an ID card configured to be carried in a wallet or purse.

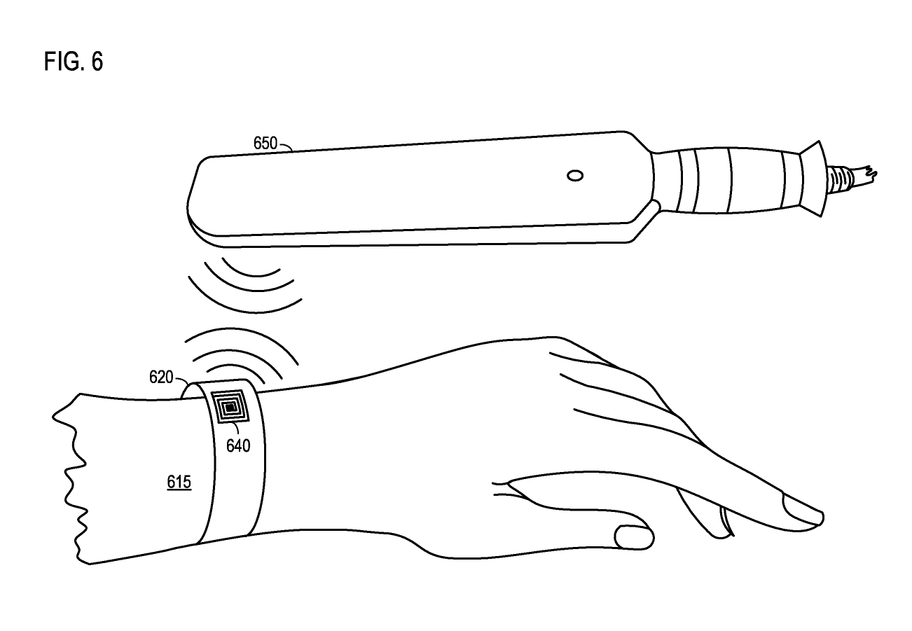

FIG. 6 illustrates a handheld RFID reader 650 being used to scan an RFID tag 640 embedded in a bracelet 620 secured to the wrist 615 of a patient. The distance at which handheld RFID reader 650 must be placed relative to RFID tag 640 in order to receive identification information may depend on the signal strength of RFID tag 640 and the sensitivity of handheld RFID reader 650. According to some embodiments, the sensitivity of handheld RFID reader 650 and/or the signal strength of RFID tag 640 may be adapted such that a relatively small distance is required. Such an approach may be useful if several RFID tags are present in a relatively confined area. Alternatively, the sensitivity of handheld RFID reader 650 and/or the signal strength of RFID tag 640 may be adapted such that identification information may be transferred over a relatively large distance. According to some embodiments, the sensitivity of handheld RFID reader 650 and/or the signal strength of RFID tag 640 may be dynamically adjusted by an operator.

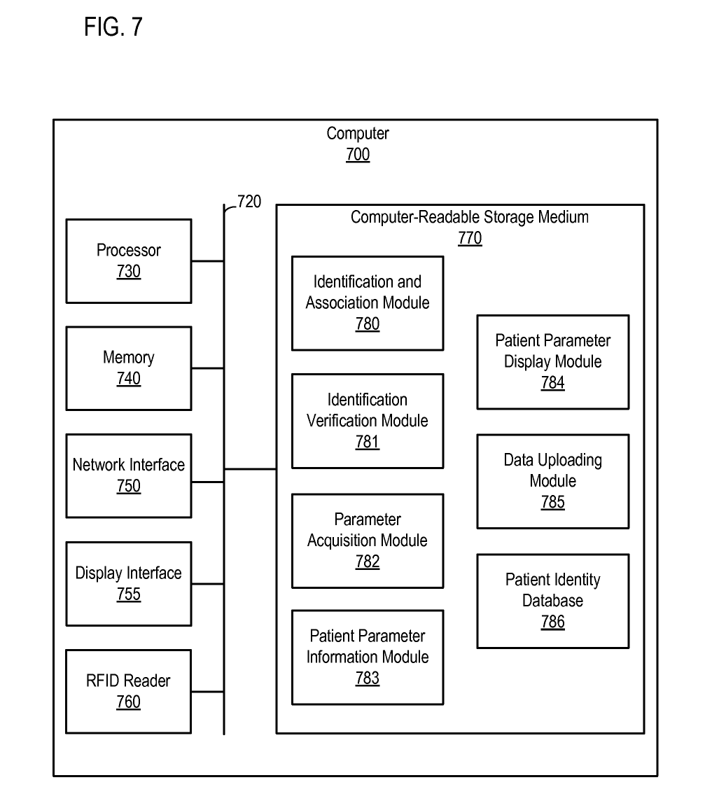

FIG. 7 illustrates a functional block diagram of a computer system 700 that may be used to monitor a patient and automatically identify a patient using RFID. As illustrated, a computer 700 may include a processor 730, memory 740 (RAM), a network interface 750, a display interface 755, and an RFID reader 760 in communication with a computer-readable storage medium 770 via bus 720. Computer-readable storage medium 770 may include one or more software modules 780-786 configured to automatically identify a patient and generate patient parameter information related to the physiological parameters of a patient. According to various embodiments, one or more of software modules 780-786 may alternatively be implemented using firmware and/or hardware. Additionally, one or more of software modules 780-786 may be joined together as a single module and/or separated into a plurality of sub-modules. Moreover, memory 740, network interface 750, display interface 755, and/or RFID reader 760 may be implemented as an external device in communication with computer 700 via a port and/or through processor 730.

RFID reader 760 may be configured to receive identification information from an RFID tag associated with a patient. Identification and association module 780 may be configured to retrieve a patient identity from a patient identity database 786 using the identification information received from the RFID tag. According to various embodiments, patient identity database 786 may be locally stored within computer 700 or accessible via network connection. Identification and association module 780 may then associate subsequent and/or prior patient parameter information with the patient identity and/or the identification information. Identification verification module 781 may be configured to request that an operator verify that the retrieved patient identity corresponds to the patient actually being monitored.

Parameter acquisition module 782 may be configured to acquire data signals relating to the physiological parameters of the patient. According to various embodiments, parameter acquisition module 782 may acquire the data signals before, after, or while receiving the identification information from the RFID tag and/or retrieving the patient identity. Patient parameter information module 783 may be configured to generate patient parameter information relating to the physiological parameters of a patient based on the acquired data signals. Identification and association module 780 may then associate the patient parameter information with the identification information and/or the patient identity. The associated patient parameter information may then be stored in memory 740 along with the identification information and/or the patient identity. Additionally or alternatively, data uploading module 785 may be used to upload the associated patient parameter information, identification information, and/or patient identity to a central management system.

A patient parameter display module 784 may prepare at least a portion of the patient parameter information, at least a portion of the identification information, and/or at least a portion of the patient identity to be displayed via display interface 755. According to some embodiments, display interface 755 may accommodate a display unit having a vertical dimension that is larger than a horizontal dimension. The display unit may be configured to display at least a portion of the patient parameter information as a waveform and an associated numerical value. For example, a waveform may graphically illustrate the heart beats of a patient along a timeline and a numerical value may indicate the current heart rate. The display unit may comprise a touch screen allowing an operator to input data and/or modify the display as needed or desired.

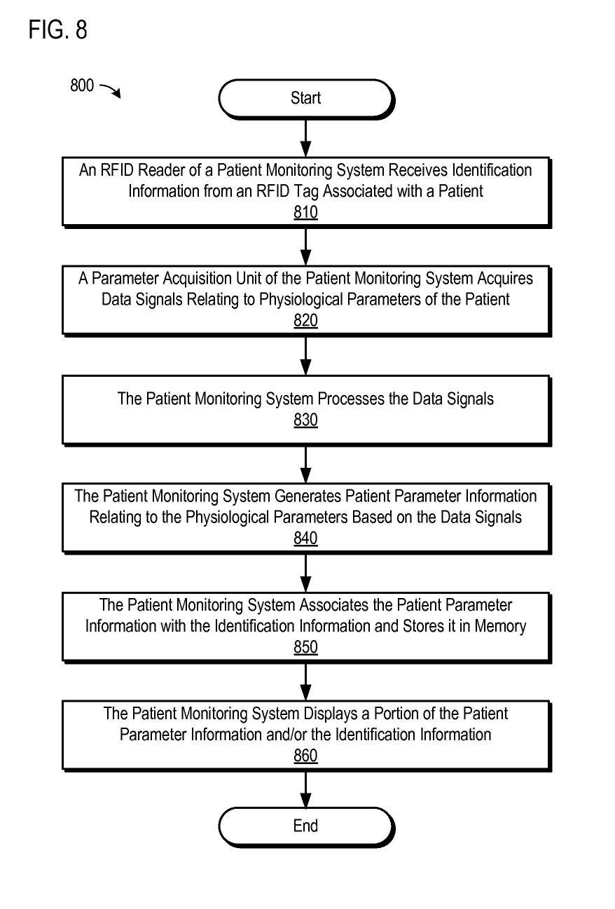

FIG. 8 illustrates a flow chart of an exemplary method 800 for automatically associating patient parameter information with identification information received via an RFID tag associated with a patient. An RFID reader of a patient monitoring system may receive identification information from an RFID tag associated with a patient, at 810. A parameter acquisition unit of the patient monitoring system may then acquire data signals relating to various physiological parameters of the patient, at 820. The patient monitoring system may then process the data signals, at 830, and generate patient parameter information relating to the physiological parameters, at 840.

The patient monitoring system may then associate the patient parameter information with the identification information and store it in a local database or in memory, at 850. According to various embodiments, the identification information and the associated patient parameter information may be subsequently retrieved. The identification information may be used to retrieve a patient identity in order to identify the patient associated with the patient parameter information. According to some embodiments, the patient parameter information and/or the identification information may be uploaded to a central management system. Additionally, the patient monitoring system may display at least a portion of the patient parameter information and/or the identification information on a display unit, at 860.

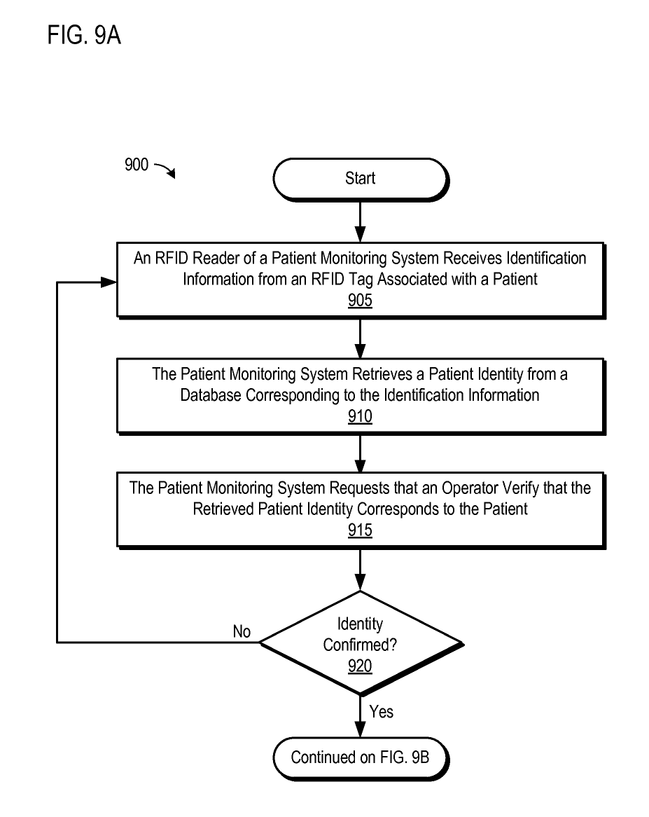

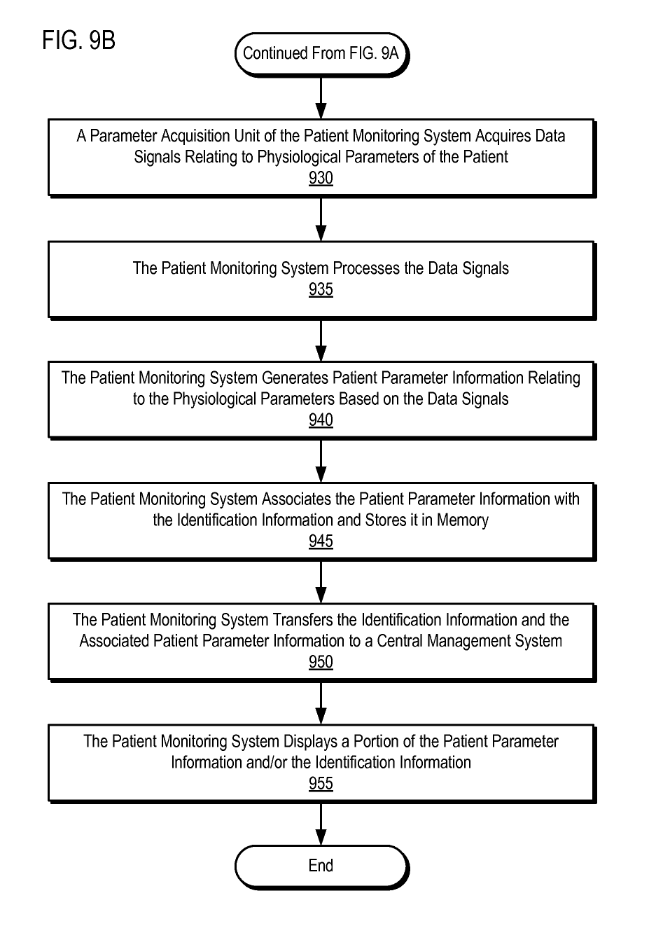

FIG. 9A illustrates a flow chart of an exemplary method 900 for automatically identifying and verifying a patient identity using an RFID tag. An RFID reader of a patient monitoring system may receive identification information from an RFID tag associated with a patient, at 905. The patient monitoring system may use the identification information to retrieve a patient identity from a database, at 910. The patient identity database may be locally stored or accessed via a network connection. The patient monitoring system may then request that an operator verify that the retrieved patient identity corresponds to the patient actually being monitored, at 915. If the identity cannot be confirmed, at 920, then the RFID reader may receive identification information again and the identification process may repeat, at 905.

Once the patient identity has been confirmed, at 920, then, as illustrated in FIG. 9B, a parameter acquisition unit of the patient monitoring system may acquire data signals relating to various physiological parameters of the patient, at 930. The patient monitoring system may then process the data signals, at 935, and generate patient parameter information relating to the physiological parameters, at 940.

The patient monitoring system may then associate the patient parameter information with the identification information and store it in a local database or in memory, at 945. Additionally or alternatively, the patient monitoring system may associate the patient parameter information with the retrieved patient identity and store it in a local database or in memory. According to some embodiments, the patient monitoring system may upload the identification information, the patient identity, and/or the associated patient parameter information to a central management system, at 950. The patient monitoring system may display at least a portion of the patient parameter information on a display unit, at 955.

At least a portion of the patient parameter information, at least a portion of the identification information, and/or at least a portion of the patient identity may be displayed on a display unit having a vertical dimension that is larger than a horizontal dimension. At least a portion of the patient parameter information may be displayed as a waveform and an associated numerical value. For example, a display may include a waveform representing the heart beats of a patient along a timeline and a numerical value indicating the current heart rate.

The display unit may comprise a touch screen allowing an operator to input data and/or modify the display as needed or desired. For example, a touch screen interface may include one or more user-selectable interface buttons, such as a function menu button, a print menu button, a setup menu button, a tools menu button, a procedures menu button, an alarms menu button, and the like. In certain embodiments, when one of the user-selected interface buttons is selected, a further set of user-selectable interface buttons (e.g., a sub-menu) may be displayed.

This disclosure has been made with reference to various exemplary embodiments including the best mode. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

Additionally, as will be appreciated by one of ordinary skill in the art, principles of the present disclosure may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

What is claimed is:

1. A patient monitoring system comprising:
a remote sensor configured to receive identification information from an electronic identification device (EID) associated with a patient;
a parameter acquisition unit configured to acquire at least one data signal relating to at least one physiological parameter of the patient;
a processing unit in communication with the parameter acquisition unit and the remote sensor, the processing unit configured to:
process the at least one data signal;
generate patient parameter information related to the at least one physiological parameter based on the at least one data signal; and
associate the patient parameter information with the identification information;
a memory unit in communication with the processing unit configured to store the associated patient parameter information; and
a display unit communicatively coupled to the processing unit, the display unit configured to display at least a portion of the patient parameter information.

2. The patient monitoring system of claim 1, wherein the remote sensor comprises a radio frequency identification (RFID) reader configured to receive identification information from an RFID tag associated with the patient.

3. The patient monitoring system of claim 1, wherein the identification information comprises a unique identification number received from an EID secured to one of a bed of the patient, clothing of the patient, a bracelet of the patient, a fixture near the patient, and an object carried by the patient.

4. The patient monitoring system of claim 1, further comprising an identification verification unit configured to:
retrieve a patient identity from a database using the identification information received from the EID; and
request verification from an operator that the retrieved patient identity information corresponds to the patient.

5. The patient monitoring system of claim 4, wherein the database is a remotely stored database and the identification verification unit retrieves the patient identity via a wireless connection.

6. The patient monitoring system of claim 4, wherein the patient identity comprises one of a first name, a last name, a portion of a birth date, and a personal identification number (PIN).

7. The patient monitoring system of claim 1, further comprising a data uploading unit configured to wirelessly transfer the identification information and the associated patient parameter information to a central management system.

8. The patient monitoring system of claim 1, wherein the display unit comprises a display area having a vertical dimension that is larger than a horizontal dimension.

9. The patient monitoring system of claim 1, wherein the display unit is configured to display at least a portion of the patient parameter information as a waveform and an associated numerical value.

10. The patient monitoring system of claim 1, wherein the at least one physiological parameter comprises one of a blood pressure, a heart rate, a temperature, a respiration rate, a venous oxygen saturation, and an electrocardiogram.

11. The patient monitoring system of claim 1, wherein the display unit comprises a touch screen display.

12. A method of monitoring a patient comprising:
receiving, via a remote sensor, identification information from an electronic identification device (EID) associated with a patient;
acquiring, via a parameter acquisition unit, at least one data signal relating to at least one physiological parameter of the patient;
processing the at least one data signal with a processing unit;
generating patient parameter information related to the at least one physiological parameter based on the at least one data signal; and
associating the patient parameter information with the identification information;
storing in a memory unit the associated patient parameter information; and
displaying, via a display unit, at least a portion of the patient parameter information.

13. The method of claim 12, wherein the remote sensor comprises a radio frequency identification (RFID) reader and the EID comprises an RFID tag.

14. The method of claim 12, wherein receiving identification information from an EID comprises receiving a unique identification number from an EID secured to one of a bed of the patient, clothing of the patient, a bracelet of the patient, a fixture near the patient, and an object carried by the patient.

15. The method of claim 12, further comprising:
retrieving a patient identity from a database using the identification information received from the EID; and
requesting verification from an operator that the retrieved patient identity information corresponds to the patient.

16. The method of claim 15, wherein the database is a remotely stored database and the identification verification unit retrieves the patient identity via a wireless connection.

17. The method of claim 15, wherein the patient identity comprises one of a first name, a last name, a portion of a birth date, and a personal identification number (PIN).

18. The method of claim 12, further comprising:
wirelessly transferring the identification information and the associated patient parameter information to a central management system.

19. The method of claim 12, wherein the display unit comprises a display area having a vertical dimension that is larger than a horizontal dimension.

20. The method of claim 12, wherein displaying at least a portion of the patient parameter information comprises displaying at least a portion of the patient parameter information as a waveform and an associated numerical value.

21. The method of claim 12, wherein the at least one physiological parameter comprises one of a blood pressure, a heart rate, a temperature, a respiration rate, a venous oxygen saturation, and an electrocardiogram.

22. The method of claim 12, wherein the display unit comprises a touch screen display.

23. A non-transitory computer-readable storage medium storing instructions that, when executed by a processor, are configured to cause the processor to perform a method, the method comprising:
receiving identification information from an EID associated with a patient;
acquiring at least one data signal relating to at least one physiological parameter of the patient;
processing the at least one data signal;
generating patient parameter information related to the at least one physiological parameter based on the at least one data signal; and
associating the patient parameter information with the identification information;
storing in a memory unit the associated patient parameter information; and
preparing at least a portion of the patient parameter information for display on a display unit.

24. The non-transitory computer-readable storage medium of claim 23, wherein the EID comprises a radio frequency identification (RFID) tag.

25. The non-transitory computer-readable storage medium of claim 23, wherein receiving identification information from an EID comprises receiving a unique identification number from an EID secured to one of a bed of the patient, patient clothing, a bracelet of the patient, a fixture near the patient, and an object carried by the patient.

26. The non-transitory computer-readable storage medium of claim 23, wherein the method further comprises:
retrieving a patient identity from a database using the identification information received from the EID; and
requesting verification from an operator that the retrieved patient identity information is correct.

27. The non-transitory computer-readable storage medium of claim 26, wherein the database is a remotely stored database and the identification verification unit retrieves the patient identity via a wireless connection.

28. The non-transitory computer-readable storage medium of claim 26, wherein the patient identity comprises one of a first name, a last name, a portion of a birth date, and a personal identification number (PIN).

29. The non-transitory computer-readable storage medium of claim 23, wherein the method further comprises:
wirelessly transferring the identification information and the associated patient parameter information to a central management system.

30. The non-transitory computer-readable storage medium of claim 23, wherein preparing at least a portion of the patient parameter information for display on a display unit comprises preparing at least a portion of the patient parameter information for display on a display unit having a vertical dimension that is larger than a horizontal dimension.

31. The non-transitory computer-readable storage medium of claim 23, wherein preparing at least a portion of the patient parameter information for display on a display unit comprises preparing at least a portion of the patient parameter information for display on a display unit as a waveform and an associated numerical value.

32. The non-transitory computer-readable storage medium of claim 23, wherein the at least one physiological parameter comprises one of a blood pressure, a heart rate, a temperature, a respiration rate, a venous oxygen saturation, and an electrocardiogram.
